(19) United States
(12) Patent Application Publication
(10) Pub. No.: US 2015/0284647 A1
(43) Pub. Date: Oct. 8, 2015

(54) WTE-PROCESSES AND RENEWABLE ENERGY OPTIMIZATION SYSTEM

(71) Applicant: Orion Enterprise International LLC, La Verne, CA (US)

(72) Inventor: Nguyen Gia Long, Ha Noi city (VN)

(21) Appl. No.: 14/518,347

(22) Filed: Oct. 20, 2014

(30) Foreign Application Priority Data
Apr. 2, 2014 (VN) 1-2014-01072

(51) Int. Cl. C10J 3/72; C12P 5/02; C10L 5/46

(52) U.S. Cl. CPC C10J 3/72; C10L 5/46; C12P 5/023; C10J 2300/1681; C10J 2300/0946; C10J 2300/0906; C10L 2200/0469; C10L 2290/544; C10L 2290/26; C10L 2290/30

(57) ABSTRACT

The present disclosure encompasses systems, machinery, and processes for handling municipal, solid waste mixtures through which this material is recycled into renewable energy. The system comprises three processes: a waste handling process, a recycling and transforming process that converts the waste into fuel, and an energy renewal process. The waste handling process includes collection, sorting, an sizing of the mixed waste; the recycling and transforming process converts collected material into RDF (refuse derived fuel) pellets, and the energy renewal process comprises decomposition of mixture rich in organic matter using biogas/anaerobic technology to collect methane gas and biogas residues, and gasification of the solid RDF pellets, converting them into renewable energy and synthetic carbon.

### CROSS REFERENCE TO RELATED APPLICATIONS

This application claims priority to Vietnamese Patent Application Serial No. VN 1-2014-01072 filed Apr. 2, 2014, the contents of which are hereby incorporated by reference in their entirety.

### FIELD

The present invention relates to the technology of handling a mixture of solid waste. More specifically, the present disclosure is directed to processes and an optimized system for disposal of mixed municipal solid waste and for recycling of those waste mixtures into renewable energy. The disclosed system is configured to optimize the number of components with simple operations and with the least amount of required equipment. That is, optimization of the system is achieved through a number of small individual steps that, collectively, provide both maximum economic and environmental benefits derived from renewed energy products.

### BACKGROUND

Domestic solid waste, including mixed municipal waste, agricultural waste, comprising mixtures of organic compounds, rags, cloth, rubber, leather and paper, plastic, food scraps, twigs, glass bottles and crockery and the like, i.e., all ungraded waste mixtures, have been a pressing problem for all mankind To handle these solid waste mixtures, there have been numerous proposed solutions employing prior technologies and processes for waste treatment systems. Most of these systems require multiple processes and multiple devices/or clusters of specialized pieces of equipment to separate the waste components in the mixture to provide sub-fractions of homogeneous waste suitable for disposal processes such as existing incinerators, biotechnology, etc.

Applicant's earlier patents, directed to “Domestic Waste Handling System”; “Waste Disposal Processing System”; and “Solid Fuel Pellets Production System,” were the basis of Vietnamese Patents No. 4631, 7338 and 9680. Even though the systems and processes of those patents have proven very useful and effective in the treatment of waste, the systems and processes described therein are not optimized to maximize high yield since they include too many pieces of equipment and processes, particularly when applied to complex waste streams and requiring separation of those streams into homogeneous waste sub-fractions.

In addition, the resources of fossil fuels (oil) have been/are being exhausted thus creating many new rules for minimizing the use of these fossil fuels.

Therefore, developing trends in the world today include not only solid waste management activities per se, but also combinations of recycling, reuse, and re-purposing for development of green energy to substitute for use of fossil fuels, thereby saving natural resources that are being depleted.

Currently, there are numerous waste treatment technology activities being considered worldwide (including pre-sorted and homogeneous waste mixtures), involving various methods of recycling waste into other products such as microbiological fertilizer, RDF pellets, recycled materials, etc., and at the same time reducing pollution, minimizing the need for landfill, as well as saving natural resources.

However, these waste treatment technology activities have also encountered numerous obstacles that hinder the use of these methods, particularly since they are too cumbersome. Consequently, the rate of burial in landfills is still alarmingly high and the renewed energy products do not offer broad market utilization. Moreover, because these waste treatment technologies rely on the use of the bulky, quickly worn-out, and damaged machinery, which requires direct operators' handling, these technologies appear to be very costly and to be ineffective for preservation of the environment.

Accordingly, there is a need for new processes and an optimized system to handle all kinds of homogeneous and heterogeneous waste mixtures, and to recycle and transform those materials into renewable energy.

### BRIEF SUMMARY

In contrast to the processes noted above, the present disclosure provides an optimized system that will increase the added value of the post-processed waste, bringing high economic value for the waste-to-energy industry in the processing, recycling and energy recovery from municipal waste. Accordingly, the present disclosure provides an optimized optimization technology for processing non-homogenous waste mixtures, and for recycling and transforming them into renewable energy.

To achieve the goals noted above, the present disclosure provides an optimized process and optimization processes to handle municipal solid waste mixtures, and to recycle those wastes into fuels and then transform those fuels into renewable energy. In one embodiment, the disclosure provides the following continuous processes:

Waste Handling Process (S1) (see FIG. 1) which includes: (a) Waste collection (1); (b) Preliminary cut large size of waste into appropriate size for further processing (2); (c) Cut-tear-and-beat the preliminary cut waste (2) into smaller pieces (3), which waste (3) then is spread evenly on a conveyor; (d) Removal of metal materials from waste (4); (e) Removal of inorganic, non-flammable materials (i.e., brick, dirt, cement and the like) (5); (f) Cut waste the second time (6) with a two-axis cutting device; and (g) Collect small and evenly desired size of waste (7);

Recycling and Transforming waste-into-fuels Process (S2) to transform waste into fuel (see FIG. 1), which includes Extracting, squeezing and compressing of small waste (7) to collect organic substance (8) from waste mixture and convert waste mixture into solid refuse derived fuel (RDF) pellets (9); and

Energy Renewal Process (S3) (see FIG. 1), which includes: (a) Decomposition of mixture rich in organic matter (8) by using biogas anaerobic technology to collect methane gas (8a) and biogas residues (8b); (b) Gasification of solid RDF pellets (9) and their conversion into renewable energy (9a) and synthetic carbon (9b).

In one embodiment, the optimization system ((100); see FIG. 2) includes: (a) A waste holding area (110) that comprises a preconfigured storage space (111), a raised waste floor (112) with a 15 degree tilt versus the horizontal inside (disposed within) the storage space (111), which raised waste floor moves vertically (j) through a hydraulic lifting mechanism (113) which is arranged under the floor (112), where the raised waste floor (112) is also built with leachate drain (114) at the edges along the floor; (b) a leachate collection tank (115) is connected to the leachate drain; and (c) a waste holding area cover (not shown here).

The disclosed system (100; see FIG. 2) may further comprises a crane equipment (120) that is arranged above the waste holding area (110) and that includes a scissor clamp (122) that can move vertically (i), and horizontally (i′), to preliminarily cut large waste as well as grip and transport waste

The disclosed system ((100); see FIG. 2) also may also comprise a waste handling module (130) that includes a two-axis cut-tear-and-beat device (131) to tear and cut waste, break up large inorganic and non-flammable materials for the first time and into small pieces, and a metal removal device (132) which is located at the end of the two-axis cut-tear-and-beat device (131).

In another aspect of this embodiment, the disclosed system ((100); see FIG. 2) may include a perforated vibrating screen (133) which is made with a net, located after the metal removal device, to remove all inorganic, non-flammable materials from the waste mixture;

In still another aspect of this embodiment, the disclosed system ((100); see FIG. 2) may include a helix screw device (134), located under the perforated vibrating screen (133) to the transport non-flammable, inorganic materials to another location outside.

In a further aspect of this embodiment, the disclosed system ((100); see FIG. 2) may include a two-axis cutting device (135), located next to the perforated vibrating screen (133) to cut waste mixture for the second time into smaller uniform sizes.

In another aspect of this embodiment, the disclosed system ((100); see FIG. 2) may include an extracting, squeezing and compressing equipment (140), located after the waste handling module (130) that is used to separate water from waste mixture, press and squeeze organic substance from waste mixture, vaporizing water and releasing heat and steam from the waste mixture, and compress and compact the remaining waste mixture into RDF pellets.

In yet another aspect of this embodiment, the disclosed system ((100); see FIG. 2) may include biogas tanks (150) that are built to convert organic rich mixture into methane gas and to collect waste biogas residue.

In still another aspect of this embodiment, the disclosed system ((100); see FIG. 2) may include a gasification system (160) added to gasify solid RDF pellets and convert them into renewable energy (syngas) and synthetic carbon (clean coal).

In other aspects of this embodiment, the disclosed system (100; see FIG. 2) may also include a first conveyor (170), located between the two-axis cut-tear-beat device (131) and the metal removal device (132), a second conveyor (180), located between the perforated vibrating screen (133) and the two-axis cutting device (135), a third conveyor (190), located between the two-axis cutting device (135) and the extracting, squeezing, compressing equipment, and a fourth conveyor (200), located between the extracting, squeezing, compressing equipment (140) and the gasification system (160).

The equipment, system, and processes disclosed herein provide an efficient approach to the handling of mixtures of municipal wastes and to their recycling into fuel and the conversion of this fuel into renewable energy. The equipment, system, and processes described achieve the advantages set forth below.

Non-homogeneous municipal waste can be handled, thereby reducing cost and processing steps.

There is no requirement for pre-grading of or into homogenous waste, such as pre-sorted plastic waste, inert waste, organic waste, etc. for the next stage of processing.

Through the extracting, squeezing and compressing, the incoming waste now can be transformed and collected as a mixture of organic materials containing 60% to 70% of biodegradable wet organic substance, which substance can be converted into methane gas, a milestone that no known equivalent technology has achieved, and solid RDF pellets are generated that contain mostly inert substances such as plastic, fibers, rags, paper, leather, rubber, etc.

In addition to the efficient handling of solid waste mixture through the described invention, the processes of the invention also generate a considerable amount of energy, e.g., in the form of methane gas and biogas residues that comes from converting organic substance into biogas, which biogas residues can be used to improve agricultural soil. Moreover, the disclosed processes, equipment, and systems provide considerable amount of output energy in the form of, e.g., syngas and synthetic coal via gasification of the RDF pellets.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 Depicts (as a flow chart) all processes and stages of handling a mixture of municipal waste, through which that mixture of municipal waste is recycled into fuel that is converted into renewable energy.

FIG. 2 Depicts (as a block diagram) the presently-disclosed system of handling the mixture of municipal waste, the recycling of that waste into fuel and the conversion of this fuel into renewable energy.

### DETAILED DESCRIPTION

The following description of the preferred embodiment(s) is merely exemplary in nature and is in no way intended to limit the invention, its application, or uses.

The disclosure provided in detail below includes preferred embodiments with attached drawings. However, it should be understood that the processes, equipment, and systems disclosed herein may be amended, modified and replaced by professionals in related technical fields as to not deviate from the scope and the nature of the invention. Therefore, the scope of the invention is hereby is defined by the attached drawings and the appended claims.

### Definitions

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication, which might be used in connection with the invention.

As used herein and in the appended claims, the singular forms “a”, “an”, and “the” include plural reference unless the context clearly dictates otherwise.

As used herein, the term “waste,” means solid municipal waste including mixture of urban waste and agricultural waste; mixtures of organic compounds, rags, cloth, rubber, leather and paper, plastic, food scraps, twigs, glass bottles, crockery etc.; particularly homogeneous, as well as untreated, non-homogeneous waste. All of these types of waste mentioned above will be handled by the processes and systems of the present disclosure.

As used herein, the term “scissor clamp” means the device consisting of multiple functional blades for clamping, cutting and moving waste.

As used herein, the phrase “cut-tear-beat” with respect to a device, means that the device comprises a cutting blade, a tearing blade, and a beating blade. This multi-functional device, positioned on a two-axis shaft for cutting solid waste, tearing and crushing bags as well as beating any and all inorganic, non-flammable materials.

As used herein, the phrase “extracting, squeezing, compressing” is understood as referring to equipment with multi-functions to separate water from waste, to press and squeeze organic substances from the waste, and to compress and compact the residues into RDF pellets.

As used herein, the phrase “the mixture of wet organic,” means a mixture of organic compounds with hydrated and soft materials/tissue that can be removed from the solid waste mixtures, excluding fibers.

As used herein, the phrase, “conversion of methane gas,” means a process of metabolizing organic mixture through a biogas technology, decomposing this mixture into methane gas in the absence of oxygen condition.

As used herein, the phrase “biogas residue,” refers to the residues obtained after the conversion of organic substance into methane through the biogas technology. The biogas residue can be used to improve agricultural soil.

As used herein, the phrase “dry solid RDF pellets” means a mixture of organic fibers, inert waste and flammable waste that are already pressed and compacted.

As used herein, the phrase “synthetic carbon” means the residues of the RDF pellets after being gasified into syngas.

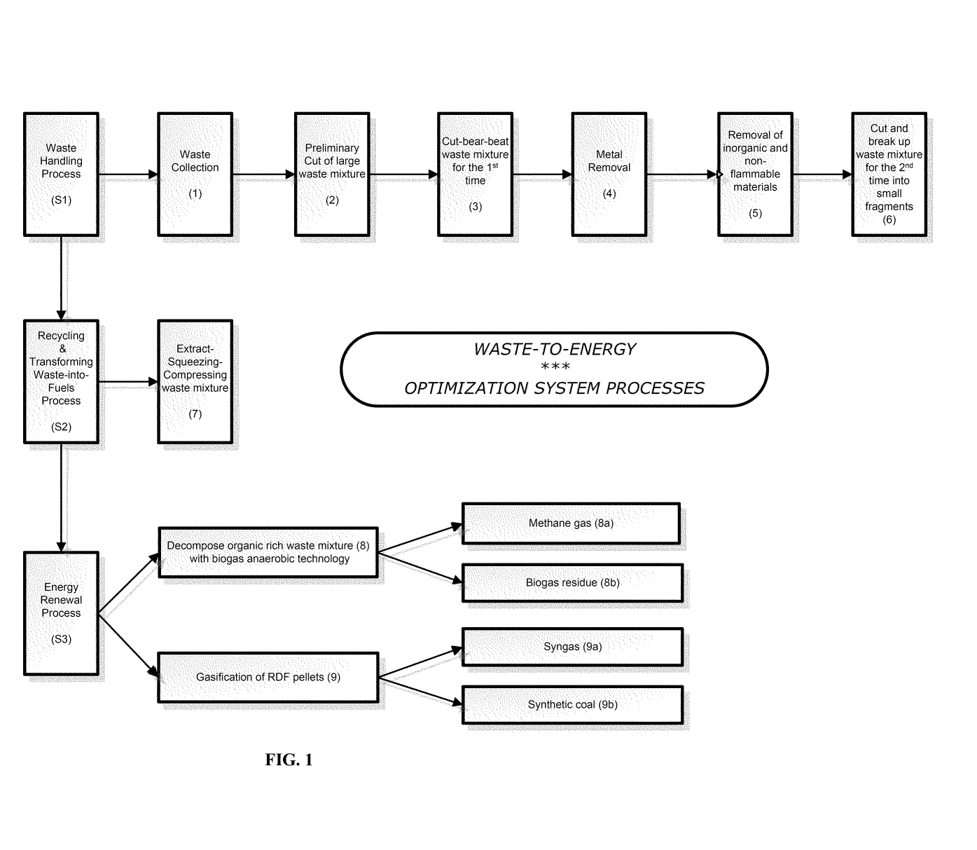

As shown in FIG. 1, the processes of handling municipal waste mixtures, recycling and transforming them into renewable energy of the invention include Waste Handling Process (S1), Recycling and Transforming waste-into-fuels Process (S2), Energy Renewal Process (S3).

The Waste Handling Process (S1) can comprise seven steps or elements (i.e., (a) through (g)): (a) Waste collection (1); (b) Preliminary cut large sizes of waste (such as mattresses, blankets, furniture, branches, waste bags, etc.) into appropriate size for further processing (2); (c) Cut-tear-and-beat the preliminary-cut waste into smaller pieces (3), and a pre-determined amount of the waste then will be spread evenly on a conveyor; (d) Removal of metal materials from waste (4); (e) Removal of inorganic, non-flammable materials (i.e., brick, dirt, cement) (5) on the moving and vibrating conveyor; (f) Cut waste the second time (6) with a two-axis cutting device; and (g) Collect small and evenly desired size of waste (7)

The Recycling and Transforming waste-into-fuels Process (S2) to transform waste into fuel comprises extracting, squeezing and compressing of small waste (7) to collect organic substance (8) from waste mixture and to convert waste mixture into solid RDF pellets (9).

The Energy Renewal Process (S3) can comprise: (a) Decomposition of the mixture rich in organic matter (8) by using biogas anaerobic technology to collect methane gas (8a) and biogas residues (8b), and (b) Gasification of solid RDF pellets (9) and convert them into renewable energy (9a) and synthetic carbon (9b).

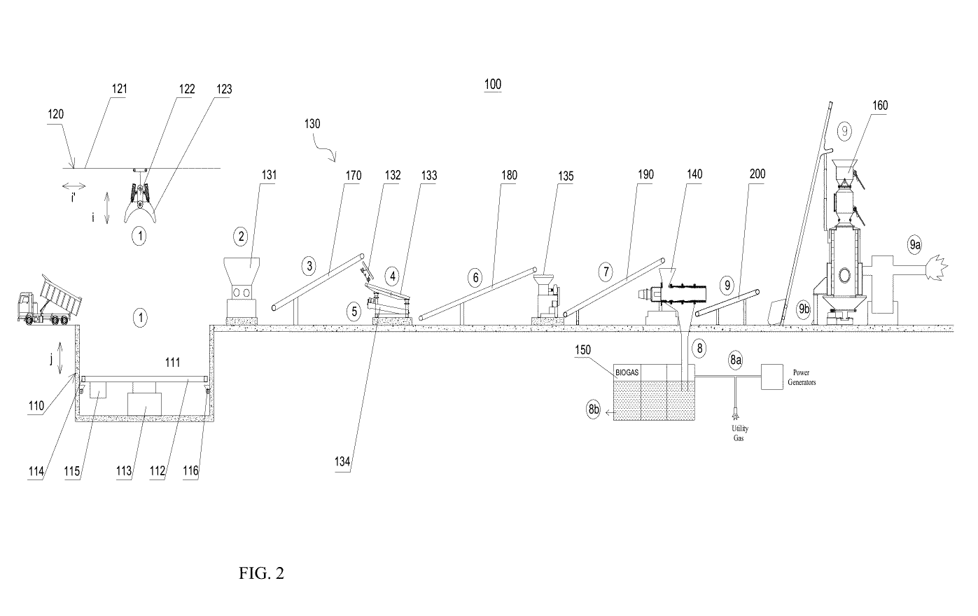

Detailed processes, equipment, and systems of the disclosure will be described below through practical operation of the invention. The following paragraphs use FIG. 2, depicting an exemplary embodiment of the disclosed system, for reference.

As shown in FIG. 2, the disclosed optimization system (100) comprises a waste holding area (110). The waste holding area may comprise the following six ((a)-(f)) elements: (a) A preconfigured storage space (111); (b) A crane equipment, located above the waste holding area (110); (c) A waste handling module (130) to separate waste and to tear and cut waste into small and even sized fragments; (d) An extracting, squeezing and compressing equipment (140) to transform waste into RDF pellets; (e) Biogas tanks (150) to convert waste into methane gas and biogas residue; and (f) A gasification system (160) to convert RDF pellets into renewable energy (syngas) and synthetic carbon (clean coal).

The waste holding area (110) may also comprise the following four elements ((g)-(k)): (g) A raised waste floor (112) that is tilted (by 15 degrees e.g.) versus the horizontal inside storage space (111); (h) The raised waste floor moves vertically (j) through a hydraulic lifting mechanism (113) which is arranged under the floor (112); (i) The raised waste floor (112) is also built with leachate drain (114) at the edges along the waste floor; (j) A leachate collection tank (115) is connected to the leachate drain (114); and (k) A waste holding area cover (not shown here).

In one embodiment of the disclosure, the waste holding area (110) also includes several block edges that are built along the perimeter of the holding area to limit the lowering of the raised waste floor (112).

In other embodiments of the disclosure, the crane equipment (120) is arranged above the waste holding area (110) to include a Frame (121) and a Scissor clamp (122) which is assembled on frame (121) and that can move vertically (i) and horizontally (i′) of frame (121).

In another embodiment of the disclosure, the scissor clamp (122) is built with several blades (123) that can preliminarily cut large waste (such as mattresses, blankets, furniture, branches, waste bags, etc.) as well as grip and transport waste.

In another embodiment of the disclosure, the waste handling module (130) may include the following five ((a)-(e)) elements: (a) Two-axis cut-tear-and-beat device (131) to tear and cut waste as well as to break up the inorganic and non-flammable materials for the first time and spread them on the conveyor; (b) A metal removal device (132) which is located at the end and is equipped with magnetic segments; (c) A perforated vibrating screen (133) made with a net, located after the metal removal device, to remove all inorganic, non-flammable materials from the waste mixture; (d) A helix screw device (134), located under the perforated vibrating screen (133) to transport non-flammable, inorganic materials to another location outside; and (e) A two-axis cutting device (135), located after perforated vibrating screen (133) to cut waste mixture for the second time into smaller uniform sizes.

The extracting, squeezing and compressing equipment (140) of the present disclosure may be located next to the waste handling sector (130). This equipment is utilized to handle smaller uniform waste sizes and to separate water from waste mixture, press and squeeze waste to collect organic substance (about 60% to 70% water and organic substance is collected here). Finally the remainder of the waste mixture is now compressed and compacted into dry, solid RDF pellets that are mostly made from inert and fiber mixture.

In certain embodiments, the extracting, squeezing and compressing equipment (140) may comprise several access chambers: waste receiving chamber, water separation chamber, wet organic substance pressing chamber, heat and steam pressing chamber, and RDF molding and pelletizing chamber.

In other embodiments, Biogas tanks (150) are built to decompose the organic-rich mixture and convert it into methane gas and to collect waste biogas residue, using the biogas technology with the absence of oxygen.

The systems of the present disclosure may also comprise a gasification system (160) that can be added to gasify solid RDF pellets and convert them into renewable energy (syngas) and synthetic carbon (clean coal).

The above processes of the optimization system are described in the Example below, using a prototype system to process one metric ton of municipal solid waste mixture based on FIG. 1 and FIG. 2.

### EXAMPLE

Referring to FIG. 1 and FIG. 2, one metric ton of waste (1) is collected and transported by truck into the waste holding area (110). In this area the waste is raised by the raised waste floor (112) via a hydraulic lifting mechanism. The raised waste floor (112) is tilted 15 degree on the side allowing leachate to flow into leachate collection tank (115).

Next, the waste mixture (1) with large sized material (such as mattresses, blankets, furniture, branches, waste bags, etc.) will primarily be cut into smaller size (2) by a scissor clamp which is mounted on the crane equipment (120) above the waste holding area (110). Waste mixture (2) is now transferred to the two-axis cut-tear-and-beat device (131) to be torn up for the first time. Components of the waste mixture with size larger than 50 mm will be cut and all inorganic, non-flammable materials (5), (such as brick, dirt, cement, glass jars, crockery) will be broken up and separated into small fragments (3). The waste mixture, with components now having reactively uniform sizes (5), is spread out on the first waste conveyor (170).

Next, the waste mixture (3) is transferred to a metal removal device (132) to separate and to collect all metal materials. The waste mixture (4) now will be transferred to a perforated vibrating screen (133) with holes of 5 mm in size. This process separates all inorganic and non-flammable materials (such as brick, dirt, cement, glass jars, crockery) which will be transferred to an outside area by the helix screw device (134).

Next, the waste mixture (6), now without the inorganic, non-flammable materials, is moved from the second waste conveyor (180) to the two-axis cutting device (135) to be cut for the second time and made into a desired evenly smaller size (7).

Next, the waste mixture (7) now is moved from the third waste conveyor (190) to the extracting, squeezing and compressing equipment (140). With this equipment, water from the waste mixture (7) will be separated and the mixture will be pressed and squeezed to collect soft degradable organic substance (8), allowing the heat and steam to escape prior to being compacted and pelletized into dry solid RDF pallets (9). According to the pilot run of the processes the volume yield in organic rich matter (8) is about 68% by weight of organic matter and water; and the volume yield in solid RDF pellets (9) is about 32% by weight of inorganic constituents.

Next, the mixture of moist organic matter (8) is transferred to the anaerobic biogas tank (150) to be converted into methane gas (8a). Methane gas (8a) can be used for power generator and biogas residues (8b) can be used for the rehabilitation of agricultural soil.

Next, the solid RDF pellets (9) now are moved from the fourth waste conveyor (200) into the gasification system to be converted into renewable energy (9a) such as syngas. Syngas can be used for power generators or power plants. Synthetic coal (9b) can be used as fuel for domestic use or for improving soil quality.

Although the invention of the present disclosure has been described through the preferred embodiments with reference to accompanying drawings, it is understood that the invention may be amended, modified and replaced under the equivalent nature of the invention by those skilled in the art and nature of the invention. Thus the scope of the invention is defined by the attached claims.

What is claimed is:

1. A system for processing waste mixtures comprising:
(a) a waste handling process;
(b) a recycling and transforming waste-into-fuels process to transform waste into fuel; and
(c) an energy renewal process;
wherein stages (a), (b), and (c) are carried out in a continuously flowing process;
whereby at least a portion of the waste mixture is recycled and at least a portion of the waste mixture is transformed into renewable energy; and
wherein said mixtures are homogeneous or heterogeneous unclassified waste mixtures.

2. The process of claim 1, wherein the waste handling process (step (a)) comprises:
(a) waste collection;
(b) preliminary cut large size of waste into appropriate size for further processing;
(c) cut-tear-and-beating the preliminary cut waste of step (b) into smaller pieces, and spreading the small pieces evenly on a conveyor;
(d) removal of metal materials from waste;
(e) removal of inorganic, nonflammable materials from the waste;
(f) cutting waste a second time with a two-axis cutting device; and
(g) collecting small and evenly desired size of waste.

3. The process of claim 2, wherein In the cut-tear-beat process (step (c)) comprises cutting, tearing and breaking up of inorganic and non-flammable materials.

4. The process of claim 2, comprising use of a plurality of waste conveyors to move waste mixture from one process step to the next process step.

5. The process of claim 2, wherein the recycling and transforming waste-into-fuels process (step (b)) comprises extracting, squeezing and compressing of small waste from step (g), to collect organic substance from the waste mixture and to convert waste mixture into solid RDF pellets; said extracting, squeezing and compressing comprising separating water from waste mixture, pressing and squeezing an organic-rich substance from the waste mixture, vaporizing water as heat and steam from the waste mixture, and compressing and compacting remaining waste mixture into RDF pellets.

6. The process of claim 6, wherein the energy renewal process comprises:
(a) decomposing the organic-rich mixture using biogas anaerobic technology to collect methane gas and biogas residues; and
(b) gasification of the solid RDF pellets and converting said pellets into renewable energy and synthetic carbon.

7. A mechanical system for carrying out the process of claim 2, the system comprising:
(a) a waste holding area;
(b) a crane disposed above the waste holding area;
(c) a waste holding module; and
(d) an extracting, squeezing, and compressing device disposed downstream of the waste handling the waste handling module and configured to operate upon the small, uniformly-sized waste material of step (g) of claim 2.

8. The mechanical system of claim 7, wherein the waste holding area comprises:
(a) a preconfigured storage space having walls and a horizontal floor;
(b) a raised waste floor disposed within and tilted with respect to the horizontal floor of the preconfigured storage space;
(c) a hydraulic lifting mechanism capable of raising the raised waste floor vertically in relation to the floor of the preconfigured storage; and
(d) a waste holding area cover.

9. The mechanical system of claim 7, wherein the crane comprises a scissor clamp capable of moving vertically and horizontally movement, cutting large components of the waste mixture; and gripping and transporting waste material.

10. The mechanical system of claim 7, wherein the waste handling module comprises:
(a) a two-axis cut-tear-and-beat device to tear and cut waste mixture a first time, and break up large inorganic and non-flammable materials for the first time into small pieces;
(b) a metal removal device disposed at the end of the two-axis cut-tear-and-beat device to remove metals from waste mixture and collect waste-mixture without metal materials;
(c) a perforated vibrating screen comprising a net, disposed after the metal removal device to remove all inorganic, non-flammable materials from the waste mixture;
(d) a helix screw device disposed under the perforated vibrating screen to transport non-flammable, inorganic materials to the exterior of the system; and
(e) a two-axis cutting device disposed next to the perforated vibrating screen to cut waste mixture for a second time into smaller pieces of uniform size.

11. The mechanical system of claim 7, further comprising biogas tanks, the conversion of the organic-rich mixture into methane gas and to collect waste biogas residue.

12. The mechanical system of claim 7, further comprising a gasification system for gasification of the solid RDF pellets and conversion of the solid RDF pellets into renewable energy (syngas) and synthetic carbon (clean coal).

13. The mechanical system of claim 7, wherein the waste storage area is submerged underground.

14. The mechanical system of claim 8, wherein the raised waste floor comprises a leachate drain at the edges of the raised waste floor and a leachate collection tank (115) connected to the leachate drain, said leachate is flowing into a tank as a consequence of the non-horizontal tilt of said raised waste floor.

15. The mechanical system of claim 14, wherein the raised waste floor is disposed at a 15 degree tilt from horizontal.

16. The mechanical system of claim 10, wherein the metal removal device comprises a magnet.

17. The mechanical system of claim 10, further comprising:
(a) a first conveyor disposed between the two-axis cut-tear-beat device and the metal removal device;
(b) a second conveyor disposed between the perforated vibrating screen and the two-axis cutting device;
(c) a third conveyor disposed between the two-axis cutting device and the extracting, squeezing, compressing equipment; and
(d) a fourth conveyor disposed between the extracting, squeezing, compressing equipment and the gasification system.
